(19) United States
(12) Patent Application Publication
(10) Pub. No.: US 2026/0092042 A1
(43) Pub. Date: Apr. 2, 2026

(54) A PROCESS FOR PREPARATION OF IPCONAZOLE & ITS INTERMEDIATES

(71) Applicant: UPL LIMITED, Maharashtra Mumbai (IN)

(72) Inventors: Anuj Kumar Mittal, Mumbai (IN); Prashant Vasant Kini, Mumbai (IN); Vilas Manikant Mukadam, Mumbai (IN); Pravin Shelkar, Mumbai (IN)

(21) Appl. No.: 19/110,260

(22) PCT Filed: Sep. 13, 2023
(86) PCT No.: PCT/IB2023/059056

§ 371 Date: Mar. 10, 2025

(30) Foreign Application Priority Data
Sep. 15, 2022 (IN) 202221052631

(51) Int. Cl. C07D 249/08; C07C 45/62; C07C 45/73; C07C 45/74; C07C 45/81; C07C 49/697

(52) U.S. Cl. CPC C07D 249/08; C07C 45/62; C07C 45/73; C07C 45/74; C07C 45/81; C07C 49/697; C07B 2200/13; C07C 2601/08

(57) ABSTRACT

The present invention relates to a process for preparation of Ipconazole and its intermediates. The present invention provides a simple, cost-effective and industrially viable process for preparation of Ipconazole and its intermediates. The process as disclosed in the present invention can be implemented for large scale production of ipconazole thereby making the process economical and environment friendly.

### FIELD OF THE INVENTION

The present invention relates to a process for preparation of Ipconazole and its intermediates.

### BACKGROUND OF THE INVENTION

Ipconazole is a triazole fungicide that is widely used for seed treatment on various crops, turfgrass, ornamental flowers and conifers. It is a systemic, broad-spectrum fungicide used to protect plants from soil borne and seed borne disease. Ipconazole is represented by formula (I) as below

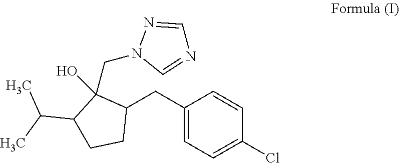 [SMILES: CC(C)C1CCC(Cc2ccc(Cl)cc2)C1(O)Cn1cncn1]

It is chemically known as 2-[(4-chlorophenyl)methyl]-5-(1-methylethyl)-1-(1H-1,2,4-triazol-1-ylmethyl)cyclopentanol.

EP0267778 discloses a process for preparation of azole derivative of formula (Ia). According to the process disclosed in said patent, an ester derivative of cyclopentane carboxylic acid of formula (a) is subjected to hydrolytic decarboxylation to obtain a cyclopentanone derivative of formula (b) which is then subjected to oxirane reaction to obtain oxirane of formula (c) which on reaction with 1,2,4-triazole provides desired azole derivative of formula (Ia). This reaction is a multistep reaction and represented as follows in Scheme I. However, this process is a complicated process.

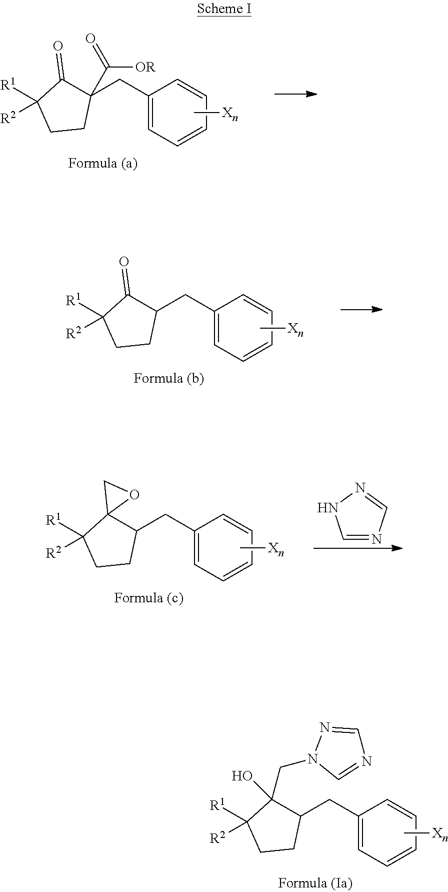

wherein R¹ and R² each independently represents a (C₁-C₅) alkyl group or a hydrogen atom; X represents a halogen atom; n represents 0, 1 or 2; and R represents a (C₁-C₅) alkyl group.

EP0329397 discloses a process for producing an azolylmethylcyclopentanol derivative by reacting 2-(4-chlorobenzyl)-5-(2-propyl)cyclopentanone with 1,2,4-triazole and sulfoxonium methylide in a polar solvent or mixture of polar solvent and an alcohol in presence of a base. The reaction is represented in below Scheme II.

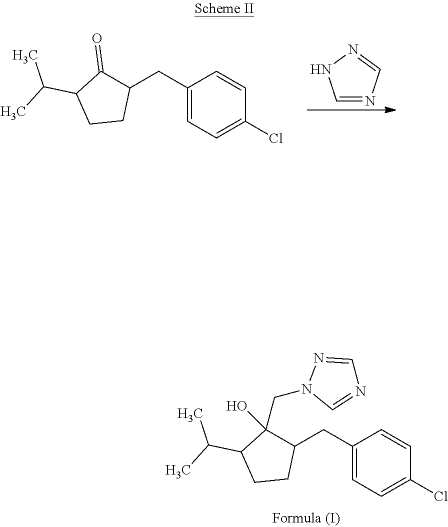 [SMILES: CC(C)C1CCC(Cc2ccc(Cl)cc2)C1=O ; c1nc[nH]n1 ; CC(C)C1CCC(Cc2ccc(Cl)cc2)C1(O)Cn1cncn1]

U.S. Pat. No. 5,466,816 discloses a process for preparing a derivative of azolylmethylcycloalkanol. This patent discloses a process for preparation of ipconazole by reacting a 2-(4-chlorobenzyl)-5-(2-propyl)cyclopentanone with 1,2,4-triazole and a sulfonium compound in presence of metal oxide and an organic solvent. However, after the completion of reaction, cooling and filtration is required to remove large amount of solid by-product generated during the reaction. Hence, it can be concluded that this process is tedious as the solid waste generated during the process is high and the process is not eco-friendly.

To meet the requirement of obtaining high purity of ipconazole, all the above process requires purification of Ipconazole using column chromatography which is less desirable for the large-scale production.

Because of the above discussed limitations, the prior art processes are not suitable for scale up at commercial level and therefore, a need exists for an industrially practical process for preparation of Ipconazole which circumvents the difficulties associated with prior art processes and simultaneously provides Ipconazole with desired high purity.

### OBJECT OF THE INVENTION

It is an object of the present invention to provide a process for preparation of Ipconazole and its intermediates, with high purity.

It is another object of the present invention to provide a simple, cost-effective and industrially viable process for preparation of Ipconazole and its intermediates.

It is another object of the present invention to provide a process for preparation of Ipconazole of formula (I) from the compound of formula (IV).

It is another object of the present invention to provide a process for preparation of a compound of formula (V)

It is yet another object of present invention to provide a crystalline form of compound of formula (IV)

It is yet another object of present invention to provide a crystalline form of Ipconazole of formula (I).

### SUMMARY OF THE INVENTION

According to an aspect of present invention, there is provided a process for preparation of a compound of formula (V)

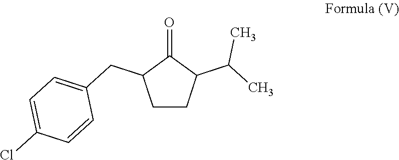 [SMILES: CC(C)C1CCC(Cc2ccc(Cl)cc2)C1=O]

comprising
a) hydrogenating a compound of formula (II) to obtain a compound of formula (III);

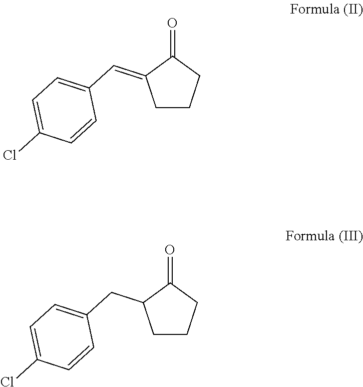 [SMILES: O=C1CCC/C1=C\c1ccc(Cl)cc1 ; O=C1CCCC1Cc1ccc(Cl)cc1]

b) reacting the compound of formula (III) with acetone to obtain a compound of formula (IV) and

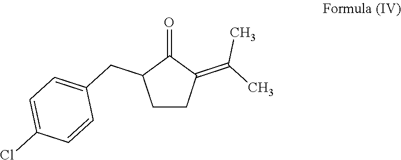 [SMILES: CC(C)=C1CCC(Cc2ccc(Cl)cc2)C1=O]

c) hydrogenating the compound of formula (IV) to obtain a compound of formula (V).

According to an aspect of present invention, there is provided a process for preparation of a compound of formula (III) comprising hydrogenating a compound of formula (II) in presence of a metal catalyst selected from Group 10 transition metal.

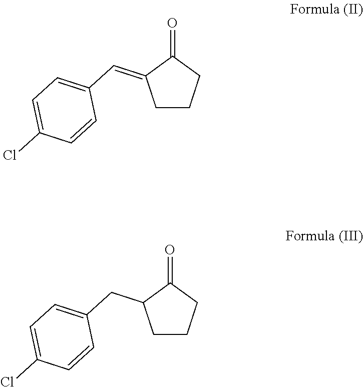 [SMILES: O=C1CCC/C1=C\c1ccc(Cl)cc1 ; O=C1CCCC1Cc1ccc(Cl)cc1]

According to yet another aspect of present invention, there is provided a process for preparation of Ipconazole of formula (I) from a compound of formula (IV).

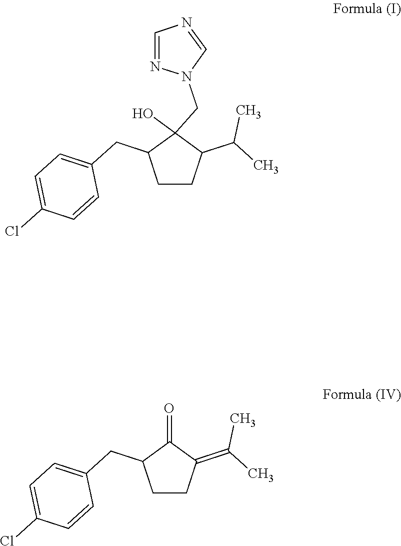 [SMILES: CC(C)C1CCC(Cc2ccc(Cl)cc2)C1(O)Cn1cncn1 ; CC(C)=C1CCC(Cc2ccc(Cl)cc2)C1=O]

According to yet another aspect of present invention, there is provided a process for preparation of Ipconazole of formula (I) from a compound of formula (IV); wherein the compound of formula (IV) is prepared by reacting the compound of formula (III) with acetone.

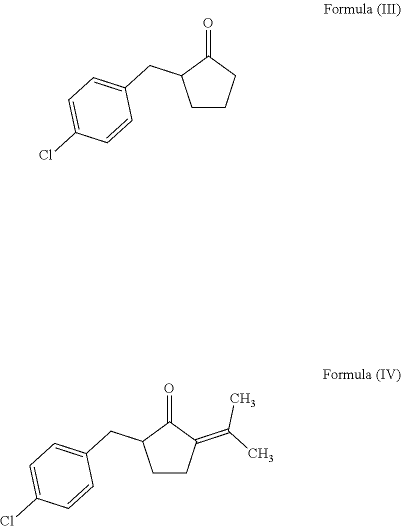 [SMILES: O=C1CCCC1Cc1ccc(Cl)cc1 ; CC(C)=C1CCC(Cc2ccc(Cl)cc2)C1=O]

According to another aspect of present invention, there is provided a crystalline form of compound of formula (IV).

In another aspect the present invention provides a crystalline form of Ipconazole of formula (I).

In another aspect, the present invention provides Ipconazole having bulk density of about 0.1 g/ml to 0.4 g/ml.

### BRIEF DESCRIPTION OF DRAWING

FIG. 1 illustrates powder X-ray diffraction (PXRD) pattern of compound of formula (IV) prepared according to present invention.

FIG. 2 illustrates powder X-ray diffraction (PXRD) pattern of Ipconazole of formula (I) prepared according to present invention.

### DETAILED DESCRIPTION OF THE INVENTION

Those skilled in art will be aware that invention described herein is subject to variations and modifications other than those specifically described. It is to be understood that the invention described herein includes all such variations and modifications. The invention also includes all such steps, features, compositions and methods referred to or indicated in this specification, individually or collectively, and any and all combinations of any two or more said steps or features.

### Definitions

For convenience, before further description of the present invention, certain terms employed in the specification, examples are described here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

The terms used herein are defined as follows.

The term “room temperature” unless stated otherwise, essentially means temperature in the range of 20-35° C.

The term “purity” means purity as determined by HPLC (“High Pressure Liquid Chromatography”).

The term “about” shall be interpreted to mean “approximately” or “reasonably close to” and any statistically insignificant variations therefrom. As used herein, the term “about” refers to a measurable value such as a parameter, an amount, a temporal duration, and the like and is meant to include variations of +1-15% or less, specifically variations of +/−10% or less, more specifically variations of +1-5% or less, even more specifically variations of +/−1% or less, and still more specifically variations of +/−0.1% or less of and from the particularly recited value, in so far as such variations are appropriate to perform in the disclosure described herein. Furthermore, it is also to be understood that the value to which the modifier “about” refers is itself specifically disclosed herein.

As used herein, the terms “comprising” “including,” “having,” “containing,” “involving,” and the like are to be understood to be open-ended, i.e., to mean including but not limited to.

The terms “preferred” and “preferably” refer to embodiments of the invention that may afford certain benefits, under certain circumstances. In an embodiment, the aspects and embodiments described herein shall also be interpreted to replace the clause “comprising” with either “consisting of” or with “consisting essentially of” or with “consisting substantially of”.

The term “crude Ipconazole” used herein means Ipconazole having low HPLC purity or obtained by the methods known to person skilled in art, having low HPLC purity.

It was observed that most of the processes known in prior art utilises column chromatography for obtaining purified Ipconazole, however due to the tedious process, use of column chromatography is not desirable at industrial scale. The inventors of present invention designed a simple process for preparation of Ipconazole having high purity without using column chromatography.

Further, the process of the present invention is based on a novel sequence of reactions which can be easily implemented at large scale production thereby making the process economical and environment friendly.

According to an aspect of present invention, there is provided a process for preparation of a compound of formula (V)

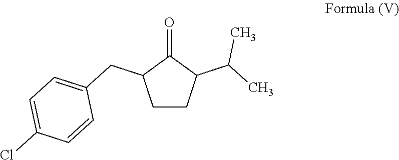 [SMILES: CC(C)C1CCC(Cc2ccc(Cl)cc2)C1=O]

comprising
a) hydrogenating a compound of formula (II) to obtain a compound of formula (III)

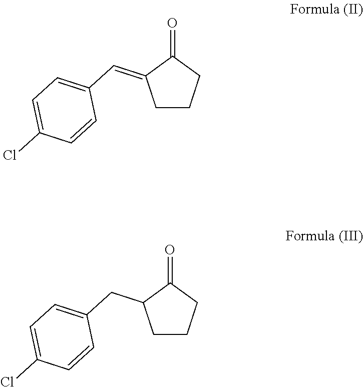 [SMILES: O=C1CCC/C1=C\c1ccc(Cl)cc1 ; O=C1CCCC1Cc1ccc(Cl)cc1]

b) reacting the compound of formula (III) with acetone to obtain a compound of formula (IV)

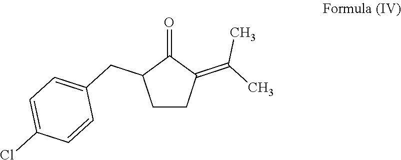 [SMILES: CC(C)=C1CCC(Cc2ccc(Cl)cc2)C1=O]

c) hydrogenating a compound of formula (IV) to obtain a compound of formula (V).

In an embodiment, the compound of formula (II) prepared by using method known in the prior art.

In an embodiment, the compound of formula (II) prepared by reacting p-chlorobenzaldehyde and cyclopentone in presence of L-proline.

According to an embodiment, the hydrogenation reaction in step a) or step c) is carried out in presence of a metal catalyst.

In an embodiment, the metal catalyst is selected from Group 10 transition metals.

In an embodiment, the Group 10 transition metal is selected from platinum, palladium, Raney nickel and the like.

According to an embodiment, the hydrogenation reaction in step a) or step c) is carried out in presence of a metal catalyst such as platinum, palladium, Raney nickel and the like.

According to an embodiment, the metal catalyst can be disposed on activated carbon or supported by alumina.

Preferably, the metal catalyst used in step a) or step c) for hydrogenation reaction. is Raney nickel.

In another embodiment the hydrogenation reaction in step a) or step c) is carried out in presence of a solvent.

The solvent used in step a) or step c) is selected from, group comprising of nitriles, alcohols, aromatic hydrocarbons or mixtures thereof.

In an embodiment, the solvent is selected from, but not limited to, nitriles like acetonitrile; C₁-C₅ alcohols like methanol, ethanol, isopropanol, n-propanol, butanol, tert-butanol; or aromatic hydrocarbons like toluene, xylene or mixtures thereof.

In an embodiment, the solvent is C₁-C₅ alcohols like methanol, ethanol, isopropanol, n-propanol, butanol, tert-butanol.

In an embodiment, the solvent is methanol.

The amount of solvent used in step a) is about 1 to 10 times with respect to compound of formula (II).

The amount of solvent used in step c) is about 1 to 10 times with respect to compound of formula (IV).

In an embodiment, the hydrogenation reaction in step a) or step c) is carried out at temperature ranging from about 0° C. to about 100° C.;

In an embodiment, the hydrogenation reaction in step a) or step c) is carried out at temperature ranging from 0° C. to 50° C.

In an embodiment, the hydrogenation reaction in step a) or step c) is carried out at temperature ranging from 10° C. to 40° C.

In an embodiment, the hydrogenation reaction in step a) or step c) is carried out at temperature ranging from 10° C. to 70° C.

In an preferred embodiment, the hydrogenation reaction in step a) or step c) is carried out at temperature ranging from about 20° C. to about 35° C.

In an embodiment, the amount of acetone used in step b) is about 1 to 10 moles with respect to compound of formula (III).

In an embodiment, the step b) of the process is carried out in presence of a base and an organic solvent.

The base used is selected from organic or inorganic base.

In an embodiment, the step b) of the process is carried out in presence of an organic base.

The organic base used may be selected from, but not limited to, alkyl compounds such as triethylamine, pyridine, pyrrolidine and the like.

According to an embodiment, the step b) of the process is carried out in presence of an inorganic base.

The inorganic base used is selected from, but not limited to, alkaline metal alkoxide such as sodium methoxide, sodium ethoxide, potassium t-butoxide and the like; or an alkaline metal hydroxide such as sodium hydroxide, potassium hydroxide, lithium hydroxide and the like.

In an embodiment, the step b) of the process is carried out in presence of sodium methoxide.

The amount of base used is in the range of 0.5 to 5 moles with respect to compound of formula (III).

The organic solvent used is selected from C₁-C₅ alcohols or mixture thereof.

The solvent C₁-C₅ alcohol is selected from methanol, ethanol, isopropanol, n-propanol, butanol, tert-butanol or mixtures thereof.

The amount of solvent used in step b) is about 1 to 10 times with respect to compound of formula (III).

In an embodiment, the step b) of the process is carried out at temperature ranging from 0° C. to 100° C.

In an embodiment, the step b) of the process is carried out at temperature ranging from 10° C. to 80° C.

In an embodiment, the step b) of the process is carried out at temperature ranging from about 25° C. to about 70° C. In an embodiment, the compound of formula (IV) obtained in step b) is purified by treatment with an alcohol.

The alcohol used in the process is selected from C₁ to C₈ alcohols like methanol, ethanol, propanol, isopropanol, n-butanol, tert-butanol and so on.

Preferably, methanol is used as solvent.

In an embodiment, the treatment of compound of formula (IV) with an alcohol comprises one or more of the processes selected from washing, making slurry of the compound of formula (IV) with the alcohol and isolating purified compound of formula (IV) or recrystallizing the compound of formula (IV) using the alcohol.

In an embodiment, there is provided a process for preparation of a compound of formula (V)

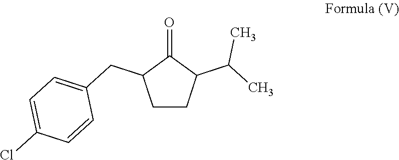 [SMILES: CC(C)C1CCC(Cc2ccc(Cl)cc2)C1=O]

comprising
a) hydrogenating a compound of formula (II) to obtain a compound of formula (III);

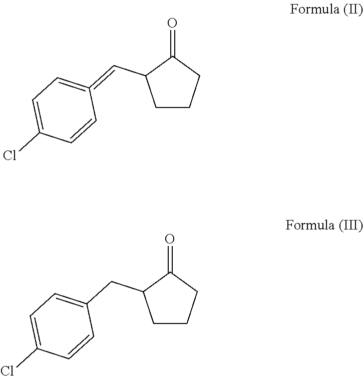

b) reacting the compound of formula (III) with acetone in presence of a base to obtain a compound of formula (IV) and

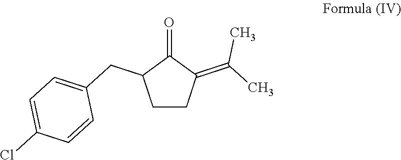 [SMILES: CC(C)=C1CCC(Cc2ccc(Cl)cc2)C1=O]

c) hydrogenating the compound of formula (IV) to obtain a compound of formula (V).

wherein the step a) or step c) is carried out in presence of Raney nickel.

In an aspect of present invention, there is provided a process for preparation of a compound of formula (III), comprising hydrogenating a compound of formula (II) in presence of a metal catalyst selected from Group 10 transition metals.

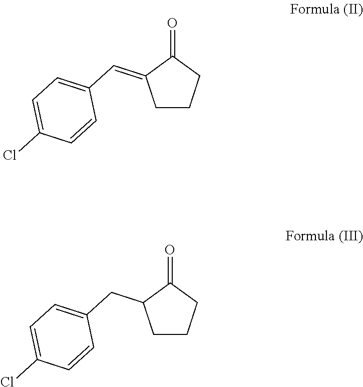 [SMILES: O=C1CCC/C1=C\c1ccc(Cl)cc1 ; O=C1CCCC1Cc1ccc(Cl)cc1]

In an embodiment, the hydrogenation of the compound of formula (II) is carried out in presence of a metal catalyst such as platinum, palladium, Raney nickel and the like.

In an embodiment, the metal catalyst can be disposed on activated carbon or supported by alumina.

Preferably, Raney nickel is used as catalyst for the hydrogenation of compound of formula (II).

In another embodiment the hydrogenation reaction is carried out in presence of a solvent selected from, but not limited to, nitriles like acetonitrile; C₁-C₅ alcohols like methanol, ethanol, isopropanol, n-propanol, butanol, tert-butanol; or aromatic hydrocarbons like toluene, xylene or mixtures thereof.

The amount of solvent used is about 1 to 10 times with respect to compound of formula (II).

In an embodiment, the hydrogenation reaction is carried out at temperature ranging from 0° C. to 100° C.

In an embodiment, the hydrogenation reaction is carried out at temperature ranging from 0° C. to 50° C.

In an embodiment, the hydrogenation reaction is carried out at temperature ranging from 10° C. to 40° C.

In an embodiment, the hydrogenation reaction is carried out at temperature ranging from about 20° C. to about 35° C.

In an aspect of present invention, there is provided a process for preparation of a compound of formula (V), comprising hydrogenating a compound of formula (IV) in presence of a metal catalyst selected from Group 10 transition metals.

In an embodiment, the Group 10 transition metal is Raney nickel.

In an embodiment, the hydrogenation reaction is carried out in presence of a solvent selected from, but not limited to, nitriles like acetonitrile; C₁-C₅ alcohols like methanol, ethanol, isopropanol, n-propanol, butanol, tert-butanol; or aromatic hydrocarbons like toluene, xylene or mixtures thereof.

In an embodiment, the solvent used is C₁ to C₅ alcohols.

The C₁ to C₅ alcohols such as methanol, ethanol, isopropanol, n-propanol, butanol, tert-butanol or mixture thereof are used.

In an embodiment, the hydrogenation reaction is carried out at temperature ranging from about 20° C. to about 35° C.

In yet another aspect of present invention, there is provided a process for preparation of Ipconazole of formula (I) using a compound of formula (IV)

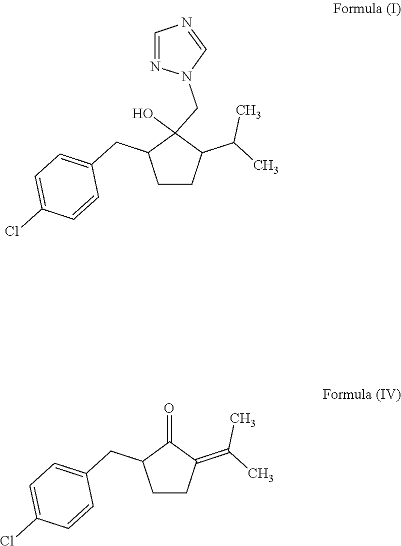 [SMILES: CC(C)C1CCC(Cc2ccc(Cl)cc2)C1(O)Cn1cncn1 ; CC(C)=C1CCC(Cc2ccc(Cl)cc2)C1=O]

In yet another aspect of present invention, there is provided a process for preparation of Ipconazole of formula (I) using a compound of formula (IV); wherein compound of formula (IV) is prepared by reacting the compound of formula (III) with acetone

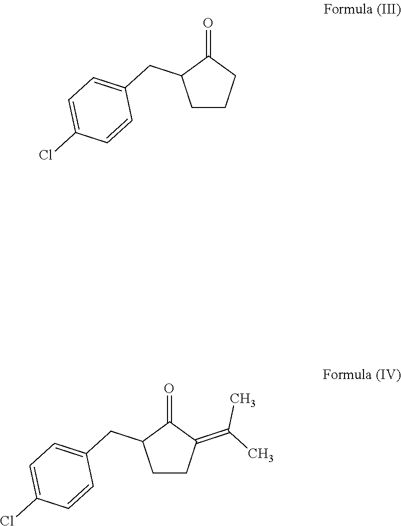 [SMILES: O=C1CCCC1Cc1ccc(Cl)cc1 ; CC(C)=C1CCC(Cc2ccc(Cl)cc2)C1=O]

According to an embodiment there is provided a process for preparation of Ipconazole of formula (I),

comprising
hydrogenating a compound of formula (IV) to obtain a compound of formula (V) and subsequently reacting the compound of formula (V) with 1,2,4-triazole or its salt to obtain Ipconazole of formula (I).

The reaction can be represented in scheme III below.

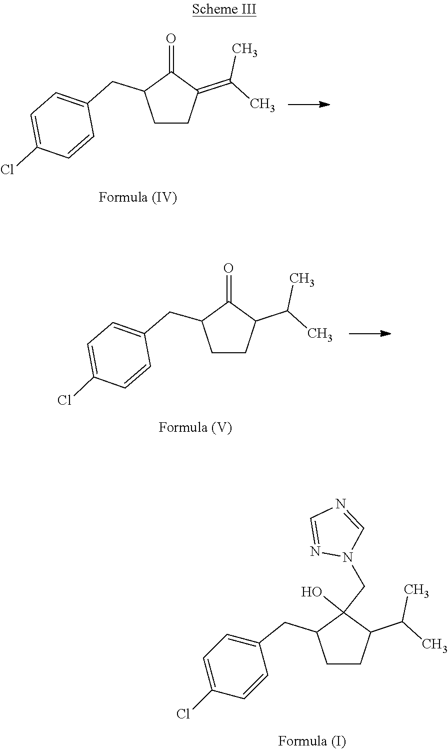 [SMILES: CC(C)=C1CCC(Cc2ccc(Cl)cc2)C1=O ; CC(C)C1CCC(Cc2ccc(Cl)cc2)C1=O ; CC(C)C1CCC(Cc2ccc(Cl)cc2)C1(O)Cn1cncn1]

The hydrogenation of compound of formula (IV) to obtain the compound of formula (V) is carried out in presence of a metal catalyst selected from group 10 transition metal.

The hydrogenation of compound of formula (IV) to obtain the compound of formula (V) is carried out in presence of a metal catalyst such as platinum, palladium, Raney nickel and the like, optionally disposed on a support.

According to an embodiment, the metal catalyst can be disposed activated carbon or supported by alumina.

In an embodiment, the metal catalyst is Raney nickel used for hydrogenation of compound of formula (IV).

In an embodiment, the hydrogenation of compound of formula (IV) is carried out at temperature ranging from 0° C. to 100° C.

In an embodiment, the reaction of compound of formula (V) with 1,2,4-triazole or its salt is carried out in presence of a base and a sulfonium halide or sulfoxonium halide to obtain Ipconazole.

The 1,2,4-triazole or its salt used is preferably in form of alkali metal salt of 1,2,4-triazole. The alkali metal salt of 1,2,4-triazole is selected from sodium 1,2,4-triazole salt, potassium 1,2,4-triazole salt, lithium 1,2,4-triazole salt, and the like.

The amount of 1,2,4-triazole or its salt used is in the range from 0.5 to 3 moles with respect to compound of formula (V).

Optionally, the sulfonium halide or sulfoxonium halide and the base are added to the reaction intermittently.

Typically, the number of the intermittent addition of the sulfonium halide or sulfoxonium halide and the base, is not limited as long as the number is sufficient to achieve a predetermined purpose. Preferably, the number of intermittent addition of the sulfonium halide or sulfoxonium halide and the base may vary from 3 to 15 times.

The sulfoxonium halide used may be selected from, but not limited to, trimethylsulfonium halide such as trimethylsulfonium iodide, trimethylsulfonium bromide, trimethylsulfonium chloride, and the like.

The sulfoxonium halide used may be selected from, but not limited to, trimethylsulfoxonium halide such as trimethylsulfoxonium iodide, trimethylsulfoxonium bromide, trimethylsulfoxonium chloride, and the like.

The amount of the sulfonium halide or sulfoxonium halide used is in the range from 0.5 to 3 moles with respect to compound of formula (V).

According to an embodiment, the base used is selected from an organic or inorganic base.

The organic base used may be selected from, but not limited to, alkyl compounds of alkali metal such as n-butyllithium; and triethylamine, pyridine, and the likes.

The inorganic base used may be selected from, but not limited to, carbonates of alkali metal such as sodium carbonate and potassium carbonate; hydroxides of alkali metal such as sodium hydroxide and potassium hydroxide; alkoxides of alkali metal such as sodium methoxide, sodium ethoxide, sodium tert-butoxide, and potassium tert-butoxide; alkali metal hydrides such as sodium hydride and potassium hydride.

The amount of base used is in the range from 0.5 to 3 moles with respect to compound of formula (V).

In an embodiment of the present invention, the reaction of compound of formula (V) with 1,2,4-triazole or its salt is carried out in presence of an organic solvent selected from, but not limited to, N-methyl-2-pyrrolidone, dimethylformamide, dimethylacetamide, dimethyl sulfoxide or its mixture with a C₁ to C₅ alcohol like methanol, ethanol, propanol, isopropanol, tert-butanol and the like.

In an embodiment of the present invention, the reaction of compound of formula (V) with 1,2,4-triazole or its salt is carried out at temperature ranging from 50 to 200° C.

In an embodiment the present process further comprises purifying Ipconazole to obtain pure Ipconazole of formula (I). The step of purifying Ipconazole comprises purifying Ipconazole with an organic solvent selected from saturated cyclic or acyclic hydrocarbons; chlorinated solvents or mixtures thereof.

In preferred embodiment, Ipconaole obtained according to the present invention is purified using suitable organic solvent.

In an embodiment, the purification process of Ipconazole with the organic solvent comprises one or more of the processes selected from washing, making slurry of the crude Ipconazole with an organic solvent selected from saturated cyclic or acyclic hydrocarbons; chlorinated solvents or mixture thereof; and isolating the pure Ipconazole of formula (I) or recrystallizing the crude Ipconazole.

The saturated cyclic or acyclic hydrocarbons used may be selected from, but not limited to, hexane, heptane, cyclohexane, methylcyclohexane and the like; saturated cyclic alcohols such as cyclopentanol, cyclohexanol, cycloheptanol and the like; or mixtures thereof.

According to an embodiment, the chlorinated solvents used are selected from but not limited to, dichloromethane, dichloroethane, chloroform and the likes; or mixture thereof.

According to an embodiment there is provided a process for preparation of Ipconazole of formula (I)

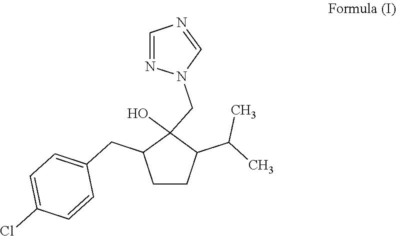 [SMILES: CC(C)C1CCC(Cc2ccc(Cl)cc2)C1(O)Cn1cncn1]

comprising
i) hydrogenating a compound of formula (II) to obtain a compound of formula (III)

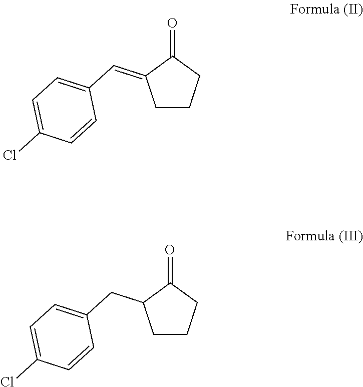 [SMILES: O=C1CCC/C1=C\c1ccc(Cl)cc1 ; O=C1CCCC1Cc1ccc(Cl)cc1]

ii) reacting a compound of formula (III) with acetone in presence of a base to obtain a compound of formula (IV)

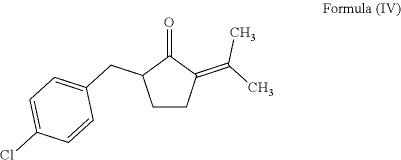 [SMILES: CC(C)=C1CCC(Cc2ccc(Cl)cc2)C1=O]

iii) hydrogenating the compound of formula (IV) to obtain a compound of formula (V) and

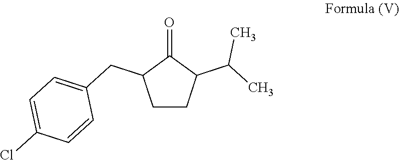 [SMILES: CC(C)C1CCC(Cc2ccc(Cl)cc2)C1=O]

iv) reacting the compound of formula (V) with 1,2,4-triazole or its salt to obtain ipconazole of formula (I).

According to yet another embodiment, there is provided a process for preparation of Ipconazole of formula (I)

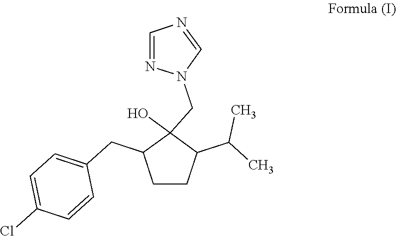 [SMILES: CC(C)C1CCC(Cc2ccc(Cl)cc2)C1(O)Cn1cncn1]

comprising
i) hydrogenating a compound of formula (II) to obtain a compound of formula (III)

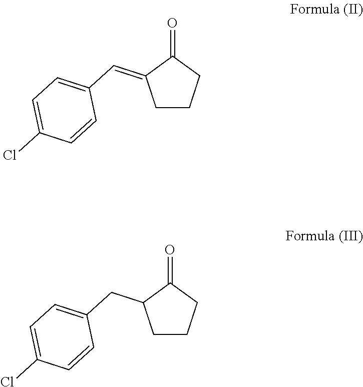 [SMILES: O=C1CCC/C1=C\c1ccc(Cl)cc1 ; O=C1CCCC1Cc1ccc(Cl)cc1]

ii) reacting the compound of formula (III) with acetone in presence of a base to obtain a compound of formula (IV)

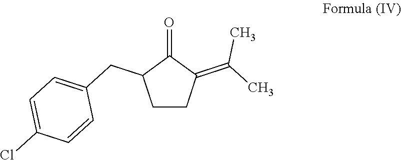 [SMILES: CC(C)=C1CCC(Cc2ccc(Cl)cc2)C1=O]

iii) hydrogenating the compound of formula (IV) to obtain a compound of formula (V)

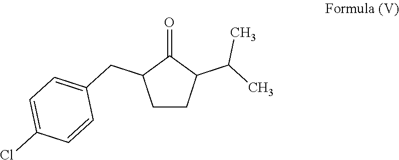 [SMILES: CC(C)C1CCC(Cc2ccc(Cl)cc2)C1=O]

iv) reacting the compound of formula (V) with 1,2,4-triazole or its salt to obtain ipconazole of formula (I).

wherein the step i) or step iii) is carried out in presence of Raney nickel.

In an embodiment, the process for preparation of Ipconazole according to present invention is illustrated by below Scheme IV.

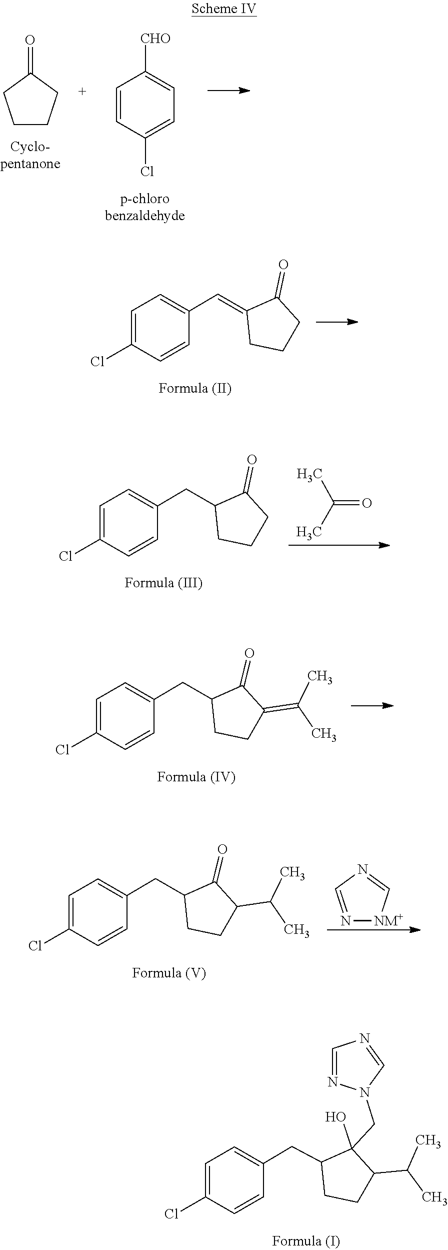

According to another aspect of present invention, there is provided a crystalline form of compound of formula (IV).

In an embodiment, there is provided a crystalline form of the compound of formula (IV) characterized by an X-ray powder diffraction pattern exhibiting at least three of the following reflections, quoted as 28±0.20 values 7.04, 12.72, 14.08, 15.28, 16.38, 18.09, 18.10, 20.87, 21.22, 22.95, 24.16, 24.67, 25.76, 26.78, 28.54, 28.70, 30.86.

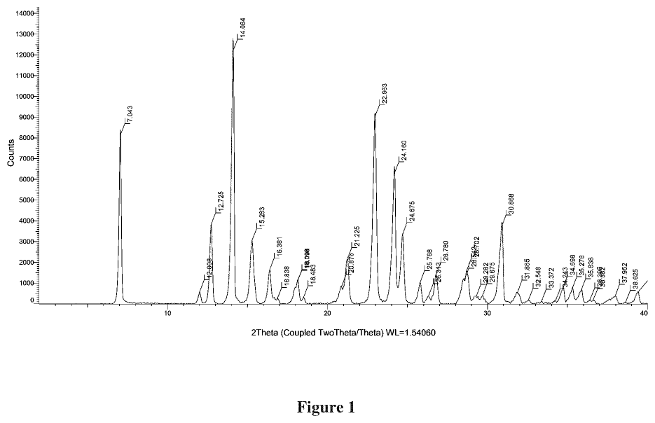

In an embodiment, there is provided a crystalline form of the compound of formula (IV) characterized by powder X-ray diffraction (PXRD) pattern represented in FIG. 1.

In another embodiment, there is provided a process for preparation of a crystalline form of compound of formula (IV).

In an embodiment the process for preparation of the crystalline form of compound of formula (IV) comprising recrystallising the compound of formula (IV) using a C₁-C₅ alcohol.

In an embodiment, C₁-C₅ alcohol is selected from methanol, ethanol, n-propanol, isopropanol or mixtures thereof.

According to another aspect of present invention, there is provided a crystalline form of Ipconazole of formula (I).

In an embodiment there is provided a crystalline form of Ipconazole of formula (I) characterized by differential scanning calorimetry (DSC) which exhibits a thermogram having an endotherm value at about 88 to 90° C.±2° C.

In another embodiment, the crystalline form of Ipconazole of formula (I) characterized by an X-ray powder diffraction pattern exhibiting at least three of the following reflections, quoted as 2θ±0.2° values 7.60, 9.84, 12.64, 14.89, 15.22, 16.46, 16.72, 18.85, 19.76, 20.35, 20.73, 21.43, 21.62, 21.87, 23.51, 25.10, 26.40, 27.57, 30.02.

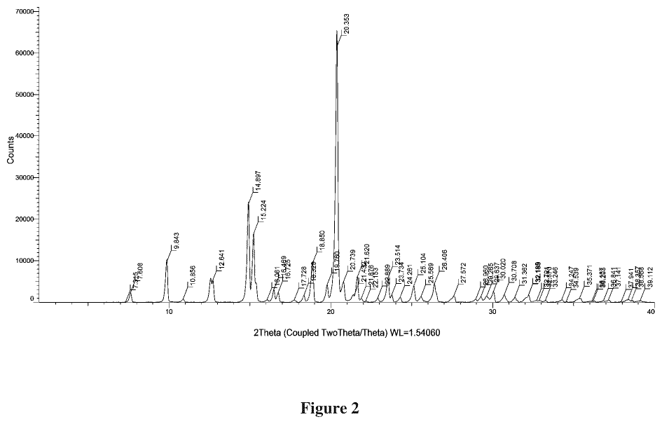

In another embodiment the crystalline form of Ipconazole of formula (I) is characterized by powder X-ray diffraction (PXRD) pattern represented in FIG. 2.

According to yet another aspect of the present invention, there is provided a process for preparation of a crystalline form of Ipconazole of formula (I).

In an embodiment the process for preparation of a crystalline form of Ipconazole of formula (I) comprising crystallising the crude Ipconazole from suitable solvent selected from a saturated cyclic or acyclic hydrocarbons; saturated cyclic alcohols; chlorinated solvents or mixture thereof.

In an embodiment, suitable solvent selected from a saturated cyclic hydrocarbons; saturated cyclic alcohols; or mixture thereof.

In an embodiment, the saturated cyclic or acyclic hydrocarbons can be selected from, but not limited to, hexane, heptane, cyclohexane, methylcyclohexane and the like; or mixtures thereof.

In an embodiment, the solvent used is a saturated cyclic hydrocarbon.

In a preferred embodiment, the solvent used is methylcyclohexane.

In an embodiment, the saturated cyclic alcohols can be selected from, but not limited to, cyclopentanol, cyclohexanol, cycloheptanol and the like; or mixtures thereof.

In an embodiment, the solvent used is mixture of saturated cyclic hydrocarbon and saturated cyclic alcohol.

In another preferred embodiment, the solvent used is mixture of cyclohexane and cyclohexanol.

In another preferred embodiment, the mixture of cyclohexane and cyclohexanol, comprises about 5% to 50% (v/v) of cyclohexanol in cyclohexane.

In another preferred embodiment, the mixture of cyclohexane and cyclohexanol, comprises about 5% to 30% (v/v) of cyclohexanol in cyclohexane.

In an embodiment, the chlorinated solvents used are selected from but not limited to, dichloromethane, dichloroethane, chloroform and the likes; or mixture thereof.

According to yet another aspect of the present invention, there is provided Ipconazole having bulk density of about 0.1 g/ml to 0.4 g/ml.

### Advantages of the Invention

1. The present invention provides Ipconazole and its intermediates, with high purity.
2. The present invention provides a simple, cost-effective and industrially viable process for preparation of Ipconazole and its intermediates.
3. The present invention avoids use of column chromatography for purification Ipconazole or its intermediates.
4. The present process is a novel sequence of steps in the process that can be implemented for large scale production of ipconazole thereby making the process economical and environment friendly.

### EXAMPLES

The following examples are presented to provide what is believed to be the most useful and readily understood description of procedures and conceptual aspects of this invention. The examples provided below are merely illustrative of the invention and are not intended to limit the same to disclosed embodiments. Variations and changes obvious to one skilled in the art are intended to be within the scope and nature of the invention.

### Methods:

X-ray powder diffraction method (XPRD) pattern was carried out on
Instrument: Bruker make 2^{nd} generation D2 Phaser Powder X-Ray diffractometer.

### Example 1: Preparation of Compound of Formula (II)

To a flask containing of p-chlorobenzaldehyde (281.25 g) and L-proline (230 g) in ethanol (1960 ml) was added cyclopentanone (185 g). The reaction mixture was stirred at 50-55° C. for 12 hours. After reaction completion, the solvent was distilled out and 5 volumes of toluene was added. The suspension obtained was stirred for 30 mins, filtrated to recover L-proline and the filtrate was washed with 1 volume of 6% aqueous sodium bicarbonate (NaHCO₃) solution. The organic layer was separated and distilled out to obtain crude compound of formula (II) (350 g) which was purified by treating with n-heptane to obtain compound of formula (II) (266 g).

### Example 2: Preparation of Compound of Formula (III)

To 225 g of compound of formula (II), 17 g of Raney Ni and 1350 ml of methanol was added. The hydrogenation reaction was carried out by charging 20 kg of hydrogen gas. The reaction mixture was maintained for 6.5 hours at 20-25° C. After completion of reaction, the reaction mixture was filtered and washed with methanol. The solvent was distilled out from filtrate to obtain 221 g of crude compound of formula (III).

### Example 3: Preparation of Compound of Formula (IV)

220 g of crude compound of formula (III) obtained according to example 2, 305.6 g of acetone and 62.64 g of sodium methoxide were taken in 1100 ml methanol and the mixture was heated to 55-60° C. for 8 hours. The solvent in reaction mixture was then distilled out at 35° C. to obtain a thick movable slurry to which 3 volumes of water was added and the pH was adjusted to 6-6.5 with acetic acid. The mixture was then extracted with dichloromethane and the organic layer washed with 7% sodium bicarbonate solution and brine. The solvent was distilled off under reduced pressure, and the resultant crude product was purified by recrystallisation with methanol to obtain 153 g of compound of formula (IV).

### Example 4: Preparation of Compound of Formula (V)

To 900 ml methanol was added 150 g of compound of formula (IV) obtained according to example 3 and 15 g Raney Nickel to obtain a reaction mixture. The hydrogenation reaction was carried out by applying 20 kg of hydrogen pressure and maintaining reaction mixture at for about 4 hours at 25-30° C. After completion of reaction, the reaction mixture was filtered and washed with methanol. The solvent was distilled out from filtrate to obtain 141 g of compound of formula (V).

### Example 5: Preparation of Ipconazole of Formula (I)

To 37.76 g of sodium 1,2,4-triazole salt was added 100 g of compound of formula (V) obtained according to example 4, and 110.7 g of N-methyl-2-pyrrolidone to obtain a reaction mixture. the reaction mixture was heated to 110° C. and then 37.56 g of sodium tert-butoxide and 103.5 g of trimethylsulfoxonium bromide were dividedly added at temperature of about 125° C. After the addition was completed, the mixture was further reacted for 1 hour. After completion of reaction, the reaction mixture was cooled to 85-90° C. and reaction mixture was distilled out to recover N-methyl-2-pyrrolidone an to obtain crude product. To the crude was added 3 volumes of water and then mixture was extracted with toluene. The solvent was removed by distillation under reduced pressure to obtain crude ipconazole of formula (I). The crude product was then crystallized from mixture of 9.09% (v/v) cyclohexanol in cyclohexane to obtain ipconazole of formula (I) having purity of 99.83%.
Bulk Density: 0.28 to 0.29 g/ml

### Example 6: Preparation of Ipconazole of Formula (I)

To 37.76 g of sodium 1,2,4-triazole salt was added 100 g of compound of formula (V) obtained according to example 4, and 110.7 g of N-methyl-2-pyrrolidone to obtain a reaction mixture. the reaction mixture was heated to 110° C. and then 37.56 g of sodium tert-butoxide and 103.5 g of trimethylsulfoxonium bromide were dividedly added at temperature of about 125° C. After the addition was completed, the mixture was further reacted for 1 hour. After completion of reaction, the reaction mixture was cooled to 85-90° C. and reaction mixture was distilled out to recover N-methyl-2-pyrrolidone an to obtain crude product. To the crude was added 3 volumes of water and then mixture was extracted with toluene. The solvent was removed by distillation under reduced pressure to obtain crude ipconazole of formula (I). The crude product was then crystallized from methylcyclohexane to obtain ipconazole of formula (I) having purity of 98.05%.

1. A process for preparation of a compound of formula (III), comprising hydrogenating a compound of formula (II) in presence of a metal catalyst selected from Group 10 transition metals to produce the compound of formula (III),

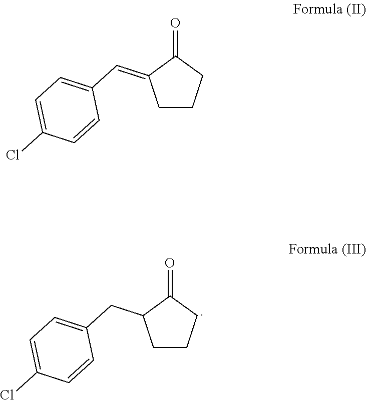 [SMILES: O=C1CCC/C1=C\c1ccc(Cl)cc1 ; O=C1CCCC1Cc1ccc(Cl)cc1]

2. The process as claimed in claim 1, wherein the metal catalyst is selected from the group consisting of platinum, palladium, or Raney nickel.

3. A process for preparation of a compound of formula (V): 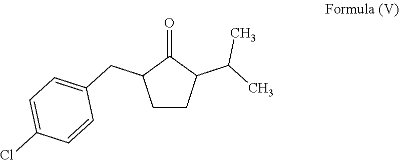 [SMILES: CC(C)C1CCC(Cc2ccc(Cl)cc2)C1=O]
comprising the steps of:
a) hydrogenating a compound of formula (II) to obtain a compound of formula (III) 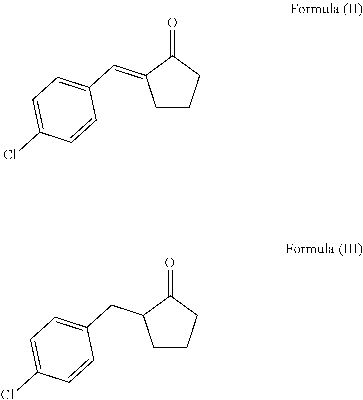 [SMILES: O=C1CCC/C1=C\c1ccc(Cl)cc1 ; O=C1CCCC1Cc1ccc(Cl)cc1]
b) reacting the compound of formula (III) with acetone to obtain a compound of formula (IV); and 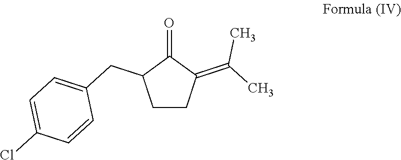 [SMILES: CC(C)=C1CCC(Cc2ccc(Cl)cc2)C1=O]
c) hydrogenating the compound of formula (IV) to obtain the compound of formula (V).

4. The process as claimed in claim 3, wherein the hydrogenation reaction in step a) or step c) is carried out in presence of a metal catalyst selected from the group consisting of platinum, palladium or Raney nickel.

5. The process as claimed in claim 3, wherein the hydrogenation reaction in step a) or step c) is carried out in presence of a solvent selected from the group consisting of of nitriles, alcohols, aromatic hydrocarbons or mixtures thereof.

6. The process as claimed in claim 3, wherein the hydrogenation reaction in step a) and step c) is carried out at a temperature ranging from about 10° C. to about 70° C.

7. The process as claimed in claim 3, wherein the compound of formula (IV) obtained in step b) of the process is purified by treatment with an alcohol.

8. The process as claimed in claim 7, wherein the alcohol is selected from the group consisting of C₁ to C₈ alcohols.

9. A crystalline form of the compound of formula (IV) characterized by an X-ray powder diffraction pattern exhibiting at least three of the following reflections, quoted as 2θ±0.2° values selected from 7.04, 12.72, 14.08, 15.28, 16.38, 18.09, 18.10, 20.87, 21.22, 22.95, 24.16, 24.67, 25.76, 26.78, 28.54, 28.70 and 30.86:

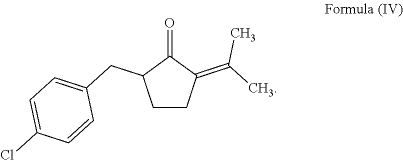 [SMILES: CC(C)=C1CCC(Cc2ccc(Cl)cc2)C1=O]

10. A process for preparation of the crystalline form of compound of formula (IV) as claimed in claim 9 comprising recrystallising the compound of formula (IV) using a C₁-C₅ alcohol.

11. A process for preparation of a compound of formula (V), comprising hydrogenating a compound of formula (IV) in presence of Group 10 transition metals selected from the group consisting of platinum, palladium and Raney nickel to produce the compound of formula (V)

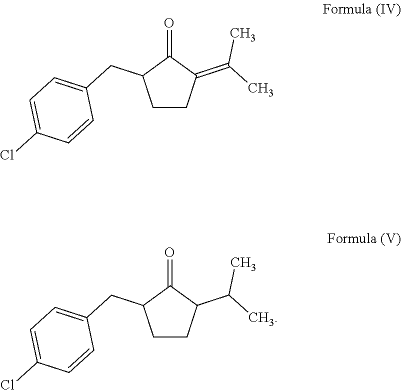 [SMILES: CC(C)=C1CCC(Cc2ccc(Cl)cc2)C1=O ; CC(C)C1CCC(Cc2ccc(Cl)cc2)C1=O]

12. A process for preparation of ipconazole of formula (I) 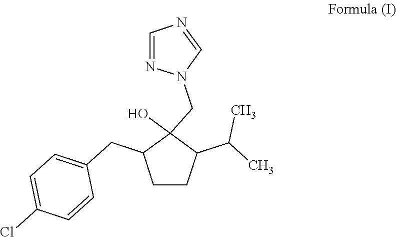 [SMILES: CC(C)C1CCC(Cc2ccc(Cl)cc2)C1(O)Cn1cncn1]
comprising the steps of:
i) hydrogenating a compound of formula (II) to obtain a compound of formula (III) 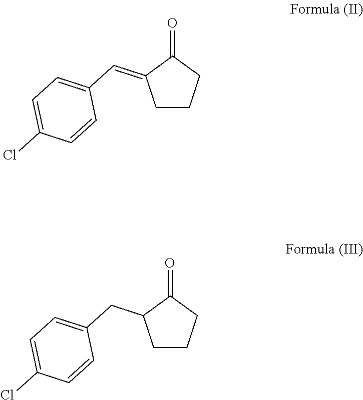 [SMILES: O=C1CCC/C1=C\c1ccc(Cl)cc1 ; O=C1CCCC1Cc1ccc(Cl)cc1]
ii) reacting a compound of formula (III) with acetone in presence of a base to obtain a compound of formula (IV), 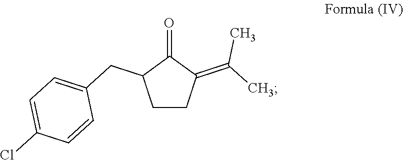 [SMILES: CC(C)=C1CCC(Cc2ccc(Cl)cc2)C1=O]
iii) hydrogenating the compound of formula (IV) to obtain a compound of formula (V), 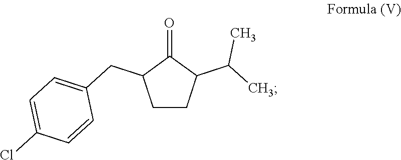 [SMILES: CC(C)C1CCC(Cc2ccc(Cl)cc2)C1=O]
iv) reacting the compound of formula (V) with 1,2,4-triazole or its salt to obtain the ipconazole of formula (I)

13. A crystalline form of Ipconazole characterized by X-ray powder diffraction pattern exhibiting at least three of the following reflections, quoted as 2θ±0.2° values selected from 7.60, 9.84, 12.64, 14.89, 15.22, 16.46, 16.72, 18.85, 19.76, 20.35, 20.73, 21.43, 21.62, 21.87, 23.51, 25.10, 26.40, 27.57, 30.02.

14. A process for preparation of a crystalline form of ipconazole of formula (I) as claimed in claim 13 comprising crystallising crude ipconazole from a solvent selected from the group consisting of saturated cyclic hydrocarbons, saturated acyclic hydrocarbons, saturated cyclic alcohols, chlorinated solvents and mixture thereof.

15. The process as claimed in claim 14, wherein the solvent is selected from the group consisting of methylcyclohexane, and a mixture of cyclohexane and cyclohexanol.

16. Ipconazole having bulk density of about 0.1 g/ml to 0.4 g/ml.
